(19) United States
(12) Patent Application Publication
(10) Pub. No.: US 2010/0228095 A1
(43) Pub. Date: Sep. 9, 2010

(54) SURGICAL RETRACTOR

(75) Inventor: Lee W. Warren, Auburn, AL (US)

Correspondence Address: Holland & Hart, LLP (LANX, Inc.);Intellectual Property Department, P.O. Box 8749, Denver, CO, 80201-8749, US

(73) Assignee: Lanx, Inc., Broomfield, CO (US)

(21) Appl. No.: 12/399,479

(22) Filed: Mar. 6, 2009

(51) Int. Cl. A61B 1/32

(52) U.S. Cl. 600/210

(57) ABSTRACT

Surgical retractors, retractor insertion tools, and methods of use are disclosed. The surgical retractor includes a first protrusion end, a second insertion end opposite the first protrusion end, and an opening extending through the retractor body from a first end opening located at the first protrusion end of the retractor body to a second end opening located at the second insertion end of the retractor body.

### BACKGROUND

Surgical retractors have long been used by surgeons as a tool for actively separating the edges of a surgical incision or wound. In so doing, surgical retractors provide improved access to an area within the body where a surgeon is operating.

One type of known surgical retractor may generally comprise a cylindrical body having an opening passing through the middle of the cylindrical body. The opposing ends of the opening through the cylindrical body are the same size and shape, making the walls of the cylindrical body parallel to one another. When one end of the cylindrical body is inserted into a wound or incision, the wound or incision area is enlarged. This provides access for surgical tools to be inserted into the wound or incision through the opening. However, the range of mobility inside the wound or incision is limited due to the parallel walls of the cylindrical body. Furthermore, sight lines are impaired once tools are extended down into the cylindrically shaped retractor. Finally, due to the curved walls of the retractor, it is difficult keep a tool stationary when it is rested against the side of the rounded retractor walls

Another type of known surgical retractor may generally comprise a conical body having an opening passing through the middle of the conical body. The opposing ends of the opening are each circular, but one end of the opening is larger than the opposite end of the opening. In application, the larger end is inserted into the wound site or incision to thereby provide a larger working area inside the wound site or incision. However, the conical body suffers from the same inability to securely rest a tool against a wall of the retractor as the conical configuration described above. Tools inserted into the wound or incision via the conical retractor also impair visibility as described above with respect to the conical retractor. Additionally, due to the concept of a lever arm, small movements of the end of the tool protruding out of the conical retractor will lead to large movements of the end of the tool located within the wound site or incision. Precise tool movements within the wound site or the incision are, therefore, hard to accomplish using a conical retractor as described above.

### SUMMARY

The present disclosure relates generally to surgical retractors and tools for aiding in the placement of surgical retractors in incisions or wound sites. The present disclosure also relates generally to methods of placing surgical retractors in incisions or wound sites.

In one embodiment disclosed herein, a surgical retractor may comprise a first protrusion end, a second insertion end opposite the first protrusion end, and an opening extending through the retractor body from a first end opening located at the first protrusion end of the retractor body to a second end opening located at the second insertion end of the retractor body. The first end opening may be larger than the second end opening. The retractor body may also taper between the first protrusion end of the retractor body and the second insertion end of the retractor body.

In another embodiment disclosed herein, a surgical retractor insertion tool may comprise a paddle-shaped spreader and a handle. The paddle-shaped spreader may comprise a first end and a second end opposite the first end. The handle may comprise a handle that is removably coupled to the paddle-shaped spreader at the first end.

In yet another embodiment disclosed herein, a method for inserting a surgical retractor into a surgical site may include a step of making an incision in a patient. The method may also comprise a step of inserting a surgical retractor insertion tool into the incision in a direction parallel to the incision. The surgical retractor insertion tool may comprise a paddle-shaped spreader and a handle. The paddle-shaped spreader may comprise a first end and a second insertion end opposite the first end, and the handle may be removably coupled to the first end. The method may also comprise a step of rotating the surgical retractor insertion tool to a position no longer parallel with the incision to widen the incision. The method may further comprise a step of removing the handle from the first end. A further step of the method may comprise inserting a retractor into the widened incision by passing the retractor over the surgical retractor insertion tool inserted in the incision.

Features from any of the above mentioned embodiments may be used in combination with one another, without limitation. In addition, other features and advantages of the instant disclosure will become apparent to those of ordinary skill in the art through consideration of the ensuing description, the accompanying drawings, and the appended claims.

### BRIEF DESCRIPTION OF THE DRAWINGS

FIG. 1 shows a perspective view of a surgical retractor according to an embodiment disclosed herein.

FIG. 2 shows a side view of the surgical retractor of FIG. 1.

FIG. 3 shows a top view of the surgical retractor of FIG. 1.

FIG. 4 shows a cross-sectional view of the surgical retractor illustrated in FIG. 1 taken along line 4-4 shown in FIG. 3.

FIG. 5 shows a perspective view of a surgical retractor according to an embodiment disclosed herein.

FIG. 6 shows a top view of a surgical retractor according to an embodiment disclosed herein

FIG. 7 shows a top view of the surgical retractor of FIG. 1 corresponding to FIG. 3 and showing tools deposited in the surgical retractor.

FIG. 8 shows a cut-away side view of the surgical retractor of FIG. 1 corresponding to FIG. 4 and showing a tool deposited in the surgical retractor.

FIG. 9 shows a side view of a surgical retractor insertion tool according to an embodiment disclosed herein.

FIG. 10 shows a front view of the surgical retractor insertion tool of FIG. 9

FIG. 11 shows a step of a method for inserting a surgical retractor into an incision made in a patient disclosed herein.

FIG. 12 shows a step of a method for inserting a surgical retractor into an incision made in a patient disclosed herein.

FIG. 13 shows a step of a method for inserting a surgical retractor into an incision made in a patient disclosed herein.

FIG. 14 shows a step of a method for inserting a surgical retractor into an incision made in a patient disclosed herein.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The instant disclosure relates generally to surgical retractors, surgical retractor insertion tools, and methods for inserting surgical retractors into an incision made in a patient. The surgical retractor disclosed herein generally employs a configuration wherein the opening of the retractor inserted into the patient is smaller than the opening protruding out of the patient. In one aspect of the surgical retractor disclosed herein, the configuration is further defined as having a curved opening inserted into the patient and a polygon-shaped opening protruding out of the patient. A curved opening is defined as one having at least one radius and at least one center point about which the curve is defined. Exemplary curves include, but are not limited to, circles, ellipses, parabolas, hyperbolic curves, pear-shaped curves, egg-shaped curves, multi-lobed curves, or any other curve. A polygon-shaped opening may have any number of sides. For example, a polygon-shaped opening may have from three to eight sides. They may be convex or non-convex. For example, a polygon is convex if any line drawn through the polygon (and not tangent to an edge or corner) meets its boundary exactly twice and non-convex if a line may be found which meets the boundary of the polygon more than twice. Exemplary polygon shapes include, but are not limited to, triangles, squares, rectangles, pentagons, hexagons, heptagons, octagons, and stars having any number of points. Curved and polygon-shaped openings may be open or closed, regular or irregular, symmetrical or asymmetrical.

This configuration provides numerous benefits over prior art retractors, including the ability to securely position a tool using a corner of the polygon-shaped opening, improved sight lines when tools are inserted into the retractor, and advantageously utilizing the concept of a lever arm, wherein larger movements of the protruding end of a tool will result in smaller movements of the end of the tool inside the incision or wound.

The retractors, surgical retractor insertion tools, and methods of this disclosure may be used for surgery in any portion of a patient's body including, but not limited to, the head, neck, chest, abdomen, joints, and other portions of the body. For example, they may be used in surgery on the human spine. Any tools may be inserted through the retractor including, but not limited to, osteotomes, burrs, reamers, forceps, scalpels, chisels, lights, suction devices, irrigation devices, implants, drivers, and other tools.

As shown in FIGS. 14, the surgical retractor 10 may generally comprise a retractor body 12 having a first protrusion end 14 and a second insertion end 16 opposite first protrusion end 14. Surgical retractor 10 may also comprise an opening 18 that extends through the entire length of retractor body 12 (ie., from first protrusion end 14 to second insertion end 16). Because opening 18 extends through the entire length of the retractor body 12, opening 18 may comprise a first end opening 20 located at first protrusion end 14 of retractor body 12 and a second end opening 22 located at second insertion end 16 of retractor body 12. First end opening 20 may be larger than second end opening 22 such that opening 18 is defined by a generally conical shape that tapers from first end opening 20 to second end opening 22. In this embodiment the retractor body has a generally constant and relatively thin wall thickness 60 such that the retractor body 12 mimics the shape of the opening 18 and is a generally conical shape that tapers from a first width 62 to a second width 64. In other embodiments, the opening 18 may define one shape while the retractor body 12 defines another, different shape. The retractor body includes a chamfer 66 to ease insertion of the retractor through an incision.

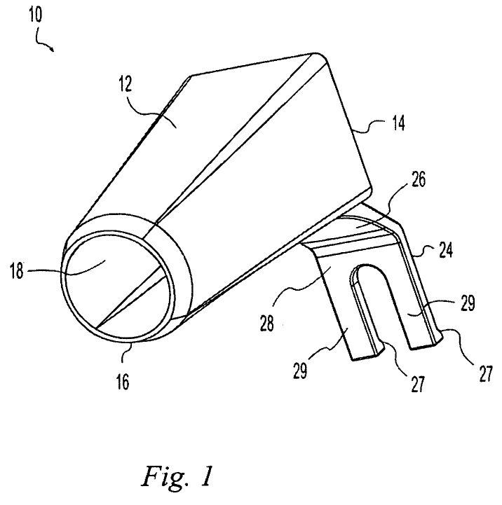

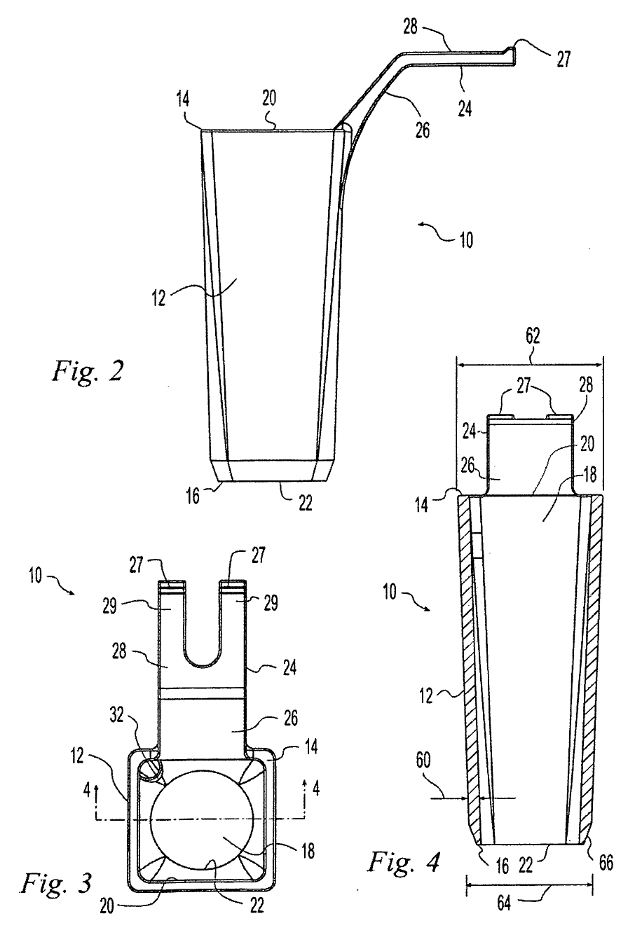

In application, surgical retractor 10 as shown in FIGS. 1-4 may be inserted into a wound or incision made in a patient to open up the wound or incision area and provide improved access to and visibility of the wound or incision area. Surgical retractor 10 may be designed such that second insertion end 16 serves as the insertion end and first protrusion end 14 serves as the portion of the surgical retractor that protrudes out of the patient. Retractor body 12 keeps the edges of the wound or incision spread apart to allow for access into the wound or incision area. That is to say, upon insertion, the edges of the wound or incision rest against the exterior of retractor body 12 and are retained apart by the retractor body 12. Once in place, opening 18, including first end opening 20 and second end opening 22, provides the pathway for viewing inside the wound or incision as well as for inserting surgical tools into the wound or incision. Such surgical tools may generally be introduced into the wound or incision by passing the tools through first end opening 20 and down opening 18 to second end opening 22.

As best seen in FIGS. 1 and 3, first end opening 20 and second end opening 22 may have specific shapes designed to improve the usefulness of-surgical retractor 10. Specifically, first end opening 20 may have a polygon shape and second end opening 22 may have a curved shape. While FIGS. 1 and 3 illustrate a square-shaped first end opening 20 and a circular shaped second end opening 22, any type of polygon or curve may be used.

While polygon shapes are described above for first end opening 20, the shape of first end opening 20 is not limited. First end opening 20 may have a circular shape or a shape utilizing both straight segments and curved segments. Likewise, while curved shapes are described for second end opening 22, the shape of second end opening is not limited. Second end opening 22 may have a polygon shape or a shape utilizing both straight segments and curved segments.

When first end opening 20 comprises a polygon shape, surgical retractor 10 may include corners in the proximity of first protrusion end 14. For example, as shown in FIG. 3, the square shape of first end opening 20 provides four corners at first protrusion end 14 of surgical retractor 10. Where second end opening 22 has a circular shape, these corners will gradually transition to the circular shape near second end 16 (as shown in e.g., FIG. 3), but corners will remain at first protrusion end 14 of surgical retractor 10.

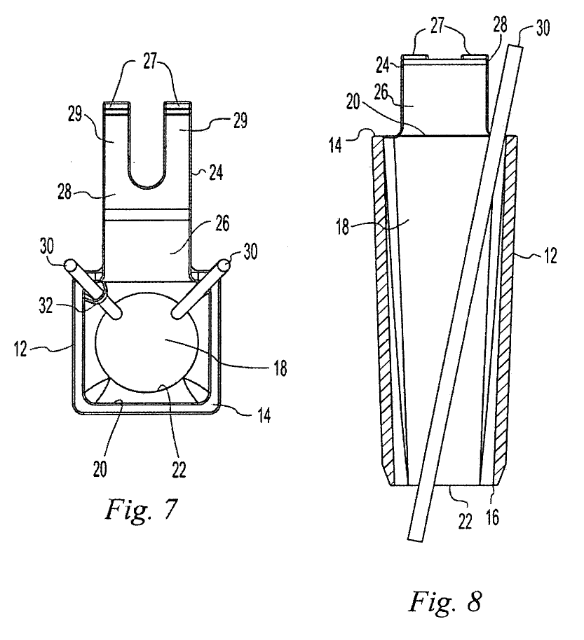

As shown in FIGS. 7 and 8, such corners may be used for resting surgical tools 30 not in use or which need to be held in a certain position while other tools are being used (such as, e.g., a light). The tools will tend to settle or be constrained in the corners such that the corners will hold tools 30 in place, unlike rounded edges which would allow tools 30 to move freely along the curved sides. To further maintain tools 30 in place, the corners may include a tool retention feature such as tool retention loop 32 near first protrusion end 14 of surgical retractor 10. Tool retention loops 32 may generally comprise loops that extend across a corner and allow tools 30 to be inserted through the loop. The size of tool retention loops 32, including how much room is provided within the loop, is not limited. Tool retention loops 32 may be large enough to accept a variety of tools having different sizes. In one aspect, tool retention loops 32 may be adjustable. For example, tool retention loops 32 may be tightened to better secure a smaller tool, or may be enlarged to accommodate a larger tool. Any number of tool retention loops 32 may be provided, including a single tool retention loop 32 or a tool retention loop 32 for every corner of the polygon-shaped first end opening 20. The tool retention loop 32 may open radially in the form of a clip that allows the tool to engage the tool retention loop 32 radially in, for example, a snap-fitting relationship.

Surgical retractor 10 may further comprise a mounting bracket 24. Mounting bracket 24 may be used to secure surgical retractor 10 to a frame or to other equipment used when surgical retractor 10 is in place in a wound or incision. Mounting bracket 24 may generally be coupled to retractor body 12 at any location along retractor body 12. In one aspect, mounting bracket 24 is coupled to retractor body 12 at a location proximate first protrusion end 14 of retractor body 12. Mounting bracket 24 may generally protrude away from retractor body 12 and also may protrude away from second end 16 of retractor body 12. Mounting bracket 24 may be coupled to retractor body 12 using any suitable mechanism, such as glue, welding, bolts, or screws. In one aspect, mounting bracket 24 may be integrally formed with retractor body 12 such that surgical retractor 10 is one unitary piece.

When first end opening 20 of surgical retractor 10 has a polygon shape, mounting bracket 24 may be coupled to surgical retractor 10 at a side of retractor body 12 formed by the polygon shape of first end opening 20 as shown in FIG. 3, for example. Alternatively, mounting bracket 24 may extend over a corner of the polygon shape of first end opening 20 as shown in FIG. 6, for example. FIGS. 1-4, 7 and 8 illustrate the configuration where mounting bracket 24 may be coupled to retractor body 12 at a side of retractor body 12 as formed by the polygon shape of first end opening 20. The polygon shape of first end opening 20 as generally shown in the FIGS. is a square. Mounting bracket 24 may be coupled to any one of the four sides of the square shape or any one of the corners. Where the polygon shape of first end opening 20 has between three and eight sides, mounting bracket 24 may be coupled to retractor body 12 on any of the three to eight sides or the associated corners between the sides. FIG. 6 illustrates the configuration where mounting bracket 24 may be coupled to retractor body 12 by extending over a corner of the polygon shape of first end opening 20.

The shape of mounting bracket 24 may be any suitable shape for allowing mounting bracket 24 to be secured to a frame or other equipment used when surgical retractor 10 is in place. As shown in FIGS. 1-8 of this disclosure, mounting bracket 24 may generally comprise an angled section 26 coupled to retractor body 12. Angled section 26 may generally protrude away from both second end 16 and retractor body 12 of surgical retractor 10. As shown in FIGS. 1-8, angled section 26 may generally be at a 45 degree angle to an axis running through opening 18 of surgical retractor 10, although other angles may also be used. Mounting bracket 24 may further comprise a transverse section 28 that is coupled directly to angled section 26. Transverse section 28 may generally be transverse to the axis running through opening 18 of surgical retractor 10. As also shown in FIGS. 1, 3, 6 and 7, transverse section 28 may comprise two arms 29 with a slot located therebetween. The slot may serve as the area where surgical retractor 10 may be secured to a frame or other equipment used in conjunction with surgical retractor 10. Both arms 29 may also include a rib or boss 27 at the end of arms 29.

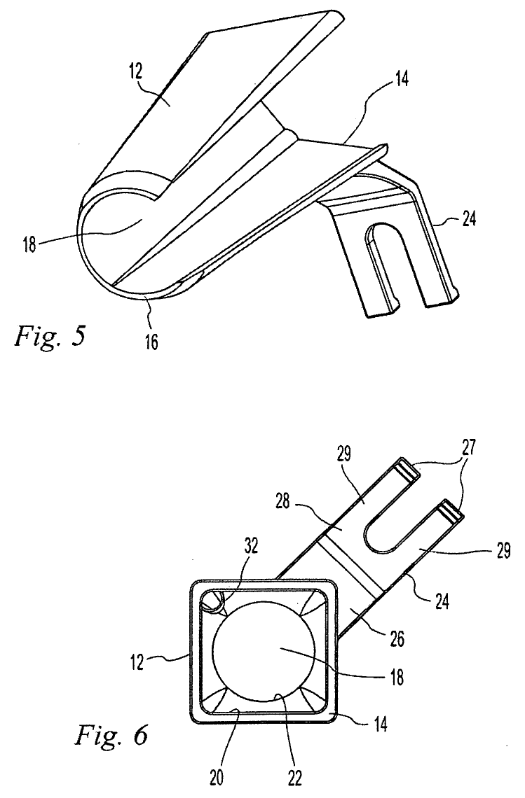

Referring now to FIG. 5, retractor body 12 may extend less than 360 degrees around opening 18. In other words, retractor body 12 may include a gap that extends from first protrusion end 14 to second insertion end 16 such that the retractor body 12 is not a closed shape. Such a configuration may further improve visibility inside the incision or the wound when surgical retractor 10 is inserted therein.

The dimensions of surgical retractor 10 are not limited and may be adjusted depending on the size of the wound or incision with which surgical retractor 10 is being used. As noted above, one limitation on dimensions may be that first end opening 20 is larger than second end opening 22. A first end opening 20 that is larger than a second end opening 22 may generally mean that retractor body 12 tapers inwardly between first end opening 20 and second end opening 22. The material of surgical retractor 10 is also not limited. In one aspect, surgical retractor 10 may comprise biocompatible material. Different parts of surgical retractor 10 may be made of the same or different materials. For example, mounting bracket 24 may be the same or a different material than the material of retractor body 12.

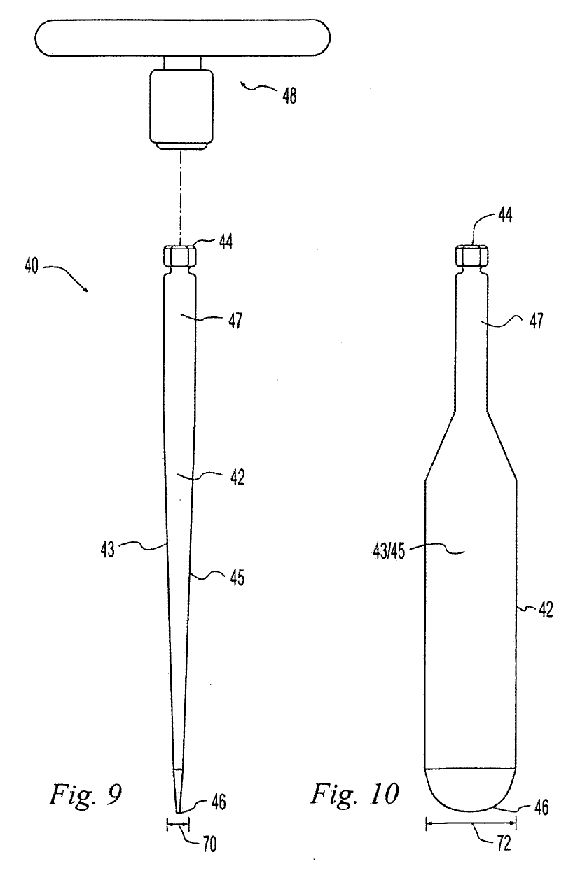

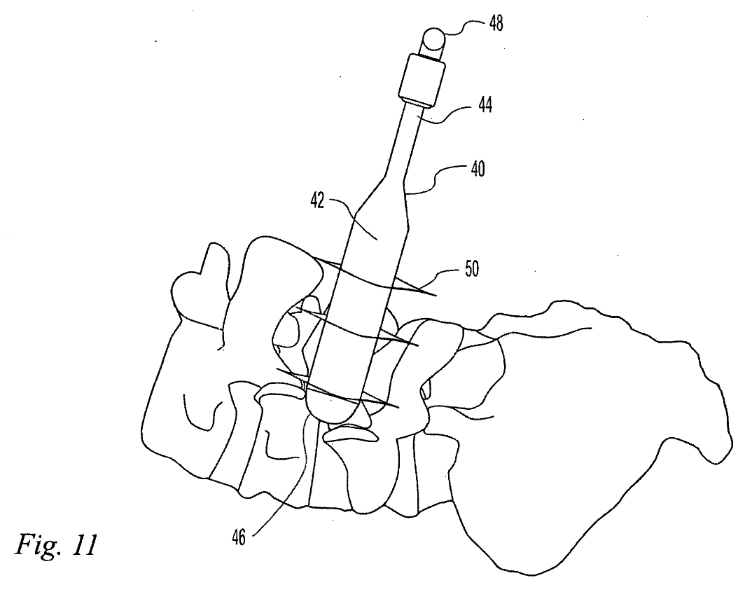

In another embodiment, a surgical retractor insertion tool that may be used to place a surgical retractor in a wound or incision is disclosed. FIGS. 9 and 10 illustrate a surgical retractor insertion tool 40. Surgical retractor insertion tool 40 may generally comprise a paddle-shaped spreader 42. Paddle-shaped spreader 42 may generally comprise a first end 44 and a second end 46 opposite first end. At first end 44 of paddle-shaped spreader 42, surgical retractor insertion tool 40 may comprise a handle 48. Handle 48 may be removably coupled to first end 44 of paddle-shaped spreader 42.

In application, surgical retractor insertion tool 40 may be inserted into a wound or incision such that surgical retractor insertion tool 40 is generally parallel with the wound or incision. Ideally, the surgical retractor insertion tool 40 is inserted such that paddle-shaped spreader 42 fits between muscle fibers (not specifically shown). Once inserted, surgical retractor insertion tool 40 may be rotated about a center axis of surgical retractor insertion tool 40. Rotation of surgical retractor insertion tool 40 may be accomplished using handle 48. Once surgical retractor insertion tool 40 is rotated to a position no longer parallel with the wound or incision, the width of paddle-shaped spreader 42 may spread apart the edges of the wound or incision, thereby making it easier to insert a surgical retractor.

The paddle shape of paddle-shaped spreader 42 may generally comprise a first broad face 43 opposite a second broad face 45, wherein the thickness 70 between first broad face 43 and second broad face 45 is small relative to the width 72 of first broad face 43 and second broad face 45. In this manner, paddle-shaped spreader 42 may resemble a paddle or oar of a boat. As shown in FIGS. 9 and 10, first broad face 43 and second broad face 45 of paddle-shaped spreader 42 may extend from second end 46 to a point short of first end 44. At this point, paddle-shaped spreader 42 may transition to a neck portion 47 that extends to first end 44. The width at neck portion 47 of paddle-shaped spreader 42 may be closer to or equal to the thickness 70 between first broad face 43 and second broad face 45. In fact, neck portion 47 of paddle-shaped spreader 42 may be cylindrical. Alternatively, first broad face 43 and second broad face 45 may extend all the way from second end 46 to first end 44 of paddle-shaped spreader 42.

The shape of first broad face 43 and second broad face 45 is not limited. As shown in FIG. 10, first broad face 43 and second broad face 45 may generally have a triangular shape at an end closest to first end 44 where paddle-shaped spreader 42 transitions from neck portion 47 to first broad face 43 and second broad face 45. First broad face 43 and second broad face 45 may then transition into a rectangular shape, wherein the edges of first broad face 43 and second broad face 45 are aligned in parallel, followed by a round-shaped edge at second end 46 of paddle-shaped spreader 42. Many other shapes and combination of shapes may also be used. For example, the entirety of first broad face 43 and second broad face 45 may have a rectangular shape, an oval shape, a diamond shape, a triangular shape, or an inverted triangular shape. First broad face 43 and second broad face 45 may also comprise any combination of the above shapes.

The thickness between first broad face 43 and second broad face 45 of paddle-shaped spreader 42 need not remain constant between first end 44 and second end 46. As shown in FIG. 9, the thickness 70 between first and second broad faces may either be maintained constant or decrease between first end 44 and second end 46. For example, the thickness 70 may remain constant near first end 44 and then transition to a constant rate of decreasing thickness as second end 46 is approached. In one aspect, the thickness 70 may decrease as second end 46 is approached such that first broad face 43 and second broad face 45 meet at second end 46 to thereby form a wedge-shaped second end 46. Such a configuration aids in inserting second end 46 into a wound or incision when using surgical retractor insertion tool 40.

Handle 48 may be removably coupled to first end 44 of paddle-shaped spreader 42. Handle 48 may be removable in order to facilitate insertion of a surgical retractor into the wound or incision after surgical retractor insertion tool 40 has been inserted and rotated to spread apart the edges of the wound or incision. Any mechanism for removably coupling handle 48 to first end 44 of paddle-shaped spreader 42 may be used. Examples include, but are not limited to, an interference fit between handle 48 and first end 44, having handle 48 screw on to first end 44, or using latches between first end 44 and handle 48 such as a ball and groove coupling. The shape of handle 48 is also not limited. As shown in FIG. 9, handle 48 may have a generally T-shape, but other shapes may be used. Handle 48, when coupled to paddle-shaped spreader 42, may be oriented in any manner with respect to paddle-shaped-spreader 42. For example, handle 48 may be perpendicular to or parallel with first broad face 43 and second broad face 45 of paddle-shaped spreader 42.

In another embodiment, a method for inserting a surgical retractor into an incision made in a patient is disclosed. The surgical retractor 10 may simply be inserted directly into an incision by pressing second insertion end 16 into the incision. The second insertion end 16 may include a chamber 66 to aid the insertion.

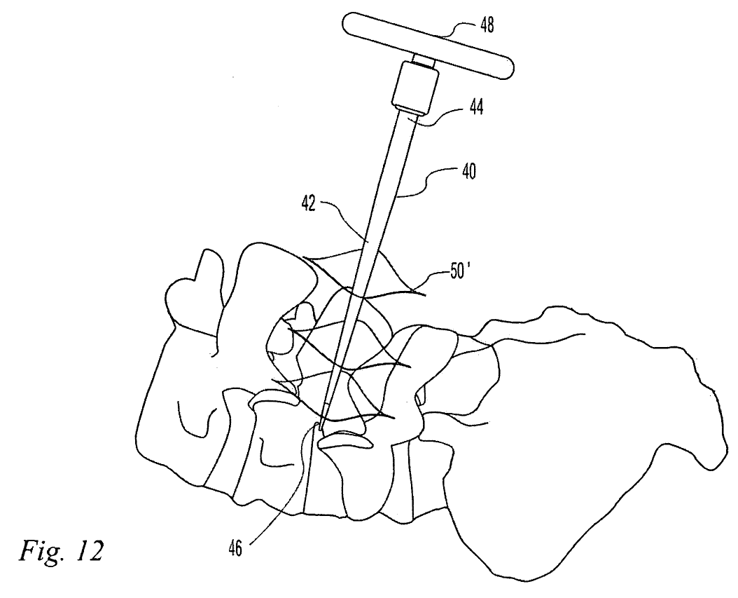

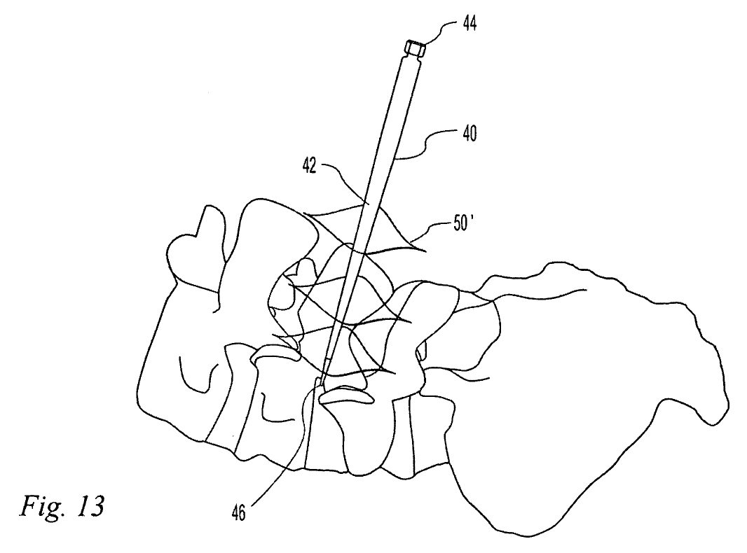

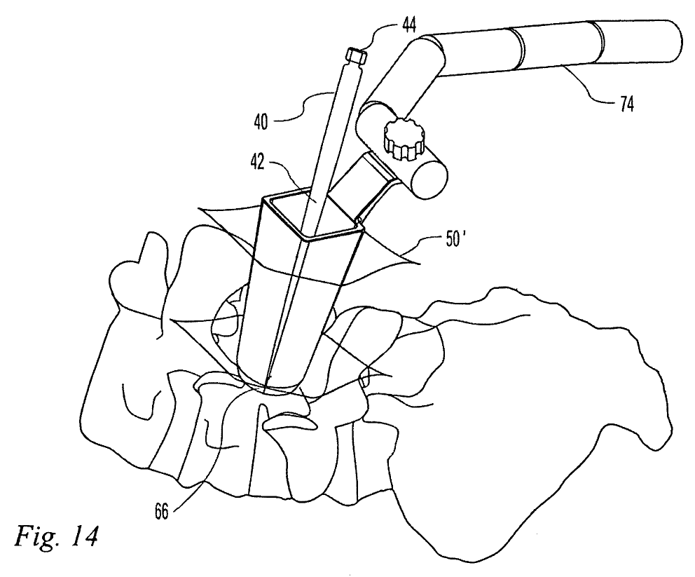

In another embodiment, a method of inserting a surgical retractor into an incision with the aid of a surgical retractor insertion tool is illustrated in FIGS. 11-14. The method may comprise a first step of making an incision 50 in a patient. The method may also comprise a step of inserting a surgical retractor insertion tool 40 into incision 50 as shown in FIG. 11. Surgical retractor insertion tool 40 may be inserted into incision 50 in a direction parallel to incision 50. The method may further comprise a step of rotating surgical retractor insertion tool 40 to a position no longer parallel with incision 50 as shown in FIG. 12. Such rotation may cause the edges of incision 50 to separate and form a widened incision 50′. The method may further comprise a step of removing a handle 48 of surgical retractor insertion tool 40 from surgical retractor insertion tool 40 as shown in FIG. 13. The method may also comprise a step of inserting a surgical retractor 10 into the widened incision 50′ as shown in FIG. 14. Surgical retractor 10 may be inserted into widened incision 50′ by passing surgical retractor 10 over surgical retractor insertion tool 40 inserted in incision 50 and rotated to create widened incision 50′.

Making an incision 50 in a patient may be a part of any type of surgery and may be accomplished according to any procedure well known to those of ordinary skill in the art. Exemplary surgeries which may entail making incision 50 may include, but are not limited to, spinal surgery or disc/pedicle surgery. Incision 50 may be made in any type of patient, including human or animal patients. In one aspect, incision 50 may be a generally straight line incision. Incision 50 may also be equal in length or longer than a width of surgical retractor insertion tool 40 so that incision 50 may accommodate the insertion of surgical retractor insertion tool 40 into incision 50.

The step of inserting surgical retractor insertion tool 40 into incision 50 may be accomplished manually or by any other suitable means for inserting surgical retractor insertion tool 40 into incision 50. Surgical retractor insertion tool 40 may be any suitable surgical retractor insertion tool. In one aspect, surgical retractor insertion tool 40 may be as described in detail above. More specifically, surgical retractor insertion tool 40 may comprise a paddle-shaped spreader 42 having a first end 44 and a second end 46 opposite first end 44 and a handle 48 removably coupled to first end 44 of paddle-shaped spreader 42. In one aspect, surgical retractor insertion tool 40 may be inserted into incision 50 in a direction parallel to incision 50. In other words, the first and second broad faces of paddle-shaped spreader 42 may be aligned in a plane parallel with incision 50. The thickness of paddle-shaped spreader 42 may be approximately equal to incision 50 such that surgical retractor insertion tool 40 may be inserted into incision 50. Surgical retractor insertion tool 40 may be inserted into incision 50 at any suitable depth that will allow surgical retractor insertion tool 40 to separate the edges of incision 50 when surgical retractor insertion tool 40 is rotated.

The step of rotating surgical retractor insertion tool 40 may be accomplished manually or by any other suitable means for rotating surgical retractor insertion tool 40 when inserted in incision 50. In one aspect, surgical retractor insertion tool 40 may be rotated by rotating handle 48 removably coupled to first end 44 of surgical retractor insertion tool 40. Surgical retractor insertion tool 40 may be rotated to any position not in parallel with incision 50. In one aspect, surgical retractor insertion tool 40 may be rotated 90 degrees to a position perpendicular to incision 50 to facilitate separation or the edges of incision 50 to create widened incision 50′.

After rotating surgical retractor insertion tool 40, handle 48 may be removed from surgical retractor insertion tool 40. Removal of handle 48 may be accomplished manually or by any other suitable means for removing handle 48 from surgical retractor insertion tool 40. Removal of handle 48 may be dictated by the manner in which handle 48 is removably coupled to surgical retractor insertion tool 40. For example, where handle 48 is removably coupled to surgical retractor insertion tool 40 by screwing handle 48 onto first end 44, handle 48 may be removed by unscrewing handle 48 from first end 44.

Once handle 48 has been removed, surgical retractor 10 may be inserted into widened incision 50′. Surgical retractor 10 may be any suitable surgical retractor. In one aspect, surgical retractor 10 is a surgical retractor as described in detail above. Surgical retractor 10 may be inserted into widened incision 50′ by sliding surgical retractor 10 down surgical retractor insertion tool 40 and into widened incision 50′. In other words, surgical retractor insertion tool 40 is passed through opening 18 of surgical retractor 10. Surgical retractor 10 may be passed over surgical retractor insertion tool 40 manually or by any other means for passing surgical retractor 10 over surgical retractor insertion tool 40 and into widened incision 50′. Surgical retractor 10 may have a size that is matched to widened incision 50′. In other words, surgical retractor 10 may have a size that will allow surgical retractor 10 to be inserted into widened incision 50. Surgical retractor 10 may have a chamfer 66 to aid in introducing second insertion end 16 into the widened incision 50′. Similarly, opening 18 of surgical retractor may have a size approximately equal to or greater than surgical retractor insertion tool 40 such that surgical retractor 10 may be passed over surgical retractor insertion tool 40. The surgical retractor 10 may then be attached to a stabilizing device such as flexible arm 74.

Once surgical retractor 10 is inserted into widened incision 50′, the edges of widened incision 50′ may be held apart by surgical retractor 10. Accordingly, the method may comprise a further step of removing surgical retractor insertion tool 40 from widened incision 50′ after surgical retractor 10 has been inserted in widened incision 50′. Alternatively, surgical retractor insertion tool 40 may remain in place for a portion or for the duration of the surgery.

While certain embodiments and details have been included herein for purposes of illustrating aspects of the instant disclosure, it will be apparent to those skilled in the art that various changes in systems, apparatus, and methods disclosed herein may be made without departing from the scope of the instant disclosure, which is defined, in part, in the appended claims. The words “including” and “having,” as used herein including the claims, shall have the same meaning as the word “comprising.”

What is claimed is:

1. A surgical retractor, comprising:
a retractor body having a first protrusion end, a second insertion end opposite the first protrusion end, and an opening extending through the retractor body from a first end opening located at the first protrusion end of the retractor body to a second end opening located at the second insertion end of the retractor body;
wherein the first end opening is larger than the second end opening and the retractor body tapers between the first protrusion end of the retractor body and the second insertion end of the retractor body.

2. The surgical retractor as claimed in claim 1 wherein the retractor body defines a wall thickness measured between an outer surface of the retractor body and an interior surface of the retractor body, surgical retractor further comprising: a chamfered portion adjacent the second insertion end of the retractor body such that the wall thickness decreases in the chamfered portion-toward the second insertion end.

3. The surgical retractor as claimed in claim 1, further comprising:
a mounting bracket coupled to the retractor body at a location proximate the first protrusion end of the retractor body.

4. The surgical retractor as claimed in claim 3, wherein the mounting bracket protrudes away from the second insertion end of the retractor body.

5. The surgical retractor as claimed in claim 1, wherein the retractor body extends less than 360 degrees around the opening.

6. The surgical retractor as claimed in claim 1, wherein the retractor body comprises biocompatible material.

7. The surgical retractor as claimed in claim 1, wherein the first end opening has a polygonal shape and the second end opening has a curved shape.

8. The surgical retractor as claimed in claim 7, wherein the polygonal shape of the first end opening is a regular polygonal shape.

9. The surgical retractor as claimed in claim 7, wherein the polygonal shape of the first end opening comprises from three to eight sides.

10. The surgical retractor as claimed in claim 8, wherein the polygonal shape comprises one or more corners and at least one of the one or more corners includes a tool retention loop proximate the first protrusion end of the retractor body.

11. The surgical retractor as claimed in claim 7, wherein the curved shape of the second end opening is circular.

12. The surgical retractor as claimed in claim 9, further comprising a mounting bracket coupled to the retractor body at one of the three to eight sides.

13. The surgical retractor as claimed in claim 7, wherein the polygonal shape comprises one or more corners, the surgical retractor further comprising a mounting bracket coupled to the retractor body at one of the one or more corners.

14. The surgical retractor as claimed in claim 1, further comprising:
A surgical retractor insertion tool including a paddle-shaped spreader with a first end and a second end opposite the first end, and a handle removably coupled to the paddle-shaped spreader at the first end, the paddle-shaped spreader being sized to pass through the opening extending through the retractor body.

15. A surgical retractor, comprising:
a retractor body having a first protrusion end, a second insertion end opposite the first protrusion end, and an opening extending through the retractor body from a first end opening located at the first protrusion end of the retractor body to a second end opening located at the second insertion end of the retractor body;
wherein the first end opening has a polygonal shape and the second end opening has a curved shape.

16. A surgical retractor insertion tool, comprising:
a paddle-shaped spreader comprising a first end and a second end opposite the first end; and
a handle removably coupled to the paddle-shaped spreader at the first end.

17. The surgical retractor insertion tool as claimed in claim 11, wherein the paddle-shaped spreader further comprises a first broad face, a second broad face opposite the first broad face, and a thickness between the first broad face and the second broad face.

18. The surgical retractor insertion tool as claimed in claim 17, wherein the thickness decreases or maintains constant from the first end of the paddle-shaped spreader to the second end of the paddle-shaped spreader.

19. The surgical spreader insertion tool as claimed in claim 17, wherein the first broad face and second broad face have a round-shaped edge at the second end of the paddle-shaped spreader.

20. The surgical spreader insertion tool as claimed in claim 17, wherein the first broad face and second broad face meet at the second end to form a wedge-shaped second end.

21. A method for inserting a surgical retractor into an incision made in a patient, the method comprising the steps of:
making an incision in a patient;
inserting a surgical retractor insertion tool into the incision in a direction parallel to the incision, wherein the surgical retractor insertion tool comprises a paddle-shaped spreader having a first end and a second insertion end opposite the first end, and a handle removably coupled to the first end of the paddle-shaped spreader;
rotating the surgical retractor insertion tool to a position no longer parallel with the incision to widen the incision;
removing the handle from the first end; and
inserting a retractor into the widened incision by passing the retractor over the surgical retractor insertion tool inserted in the incision.

22. The method of inserting a surgical retractor into an incision made in a patient as claimed in claim 21, wherein the surgical retractor insertion tool is rotated 90 degrees to a position perpendicular to the incision.

23. The method of inserting a surgical retractor into an incision made in a patient as claimed in claim 21, further comprising the step of removing the surgical retractor insertion tool from the incision after the retractor has been inserted into the incision.
